(19) United States
(12) Patent Application Publication
(10) Pub. No.: US 2016/0325004 A1
(43) Pub. Date: Nov. 10, 2016

(54) HYDROXYL GENERATION AND/OR OZONE REDUCTION SYSTEM AND METHOD

(71) Applicant: ESL Air, Inc., Kirkland, WA (US)

(72) Inventor: G. Eric Engstrom, Kirkland, WA (US)

(21) Appl. No.: 14/704,758

(22) Filed: May 5, 2015

(51) Int. Cl. A61L 9/20; B01J 19/12; B01J 19/00; B01D 53/00

(52) U.S. Cl. CPC A61L 9/20; B01D 53/007; B01J 19/123; B01J 19/0006; B01J 2219/0801; B01J 2219/1203; B01J 2219/0875

(57) ABSTRACT

System, methods and storage medium associated with generation of hydroxyl and/or reduction of ozone are disclosed. In embodiments, a system may include one or more front end sensors disposed at an input end to measure attributes of an input air stream; an hydroxyl generator/ozone reducer to receive the input air stream and use the input air stream to generate an output air stream with hydroxyl and/or reduced amount of ozone; and one or more back end sensors disposed at an output end to measure attributes of the output air stream. The system may further include a controller to control the hydroxyl generator/ozone reducer based at least in part on readings of the one or more front end sensors and the one or more back end sensors. Other embodiments may be described and/or claimed.

### TECHNICAL FIELD

The present disclosure relates to the field of air quality. More particularly, the present disclosure relates to a hydroxyl generation and/or ozone reduction system.

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

Air quality is of increasing interest to many, in particular, with 2.5 micron volatile organic compound (VOC) and excessive ozone levels. In the case of ozone, there is a great deal of evidence to show that ground level ozone can harm lung function and irritate the respiratory system. Exposure to ozone and the pollutants that produce it is linked to premature death, asthma, bronchitis, heart attack, and other cardiopulmonary problems. On the other hand, hydroxyls have been called “Mother Nature's Broom” because of their ability to clean air including the removal of ozone.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates a block diagram of a hydroxyl generation and/or ozone reduction system, according to various embodiments.

FIG. 2 illustrate the hydroxyl generator/ozone reducer of FIG. 1 in further detail, according to various embodiments.

FIGS. 3-7 illustrate the at least one ultraviolet light source of the hydroxyl generator/ozone reducer of FIGS. 1 and 2, according to various embodiments.

FIG. 8 illustrates an example process for generating hydroxyl and/or reducing ozone, according to the disclosed embodiments.

FIG. 9 illustrates an example storage medium having instructions configured to cause a controller to practice the process of FIG. 8, according to various embodiments.

### DETAILED DESCRIPTION

System, methods and storage medium associated with generation of hydroxyl and/or reduction of ozone are disclosed. In embodiments, a system may include one or more front end sensors disposed at an input end to measure attributes of an input air stream; a hydroxyl generator/ozone reducer to receive the input air stream, and use the input air stream to generate an output air stream with increased hydroxyls and/or a reduced amount of ozone; and one or more back end sensors disposed at an output end to measure attributes of the output air stream. The system may further include a controller to control the hydroxyl generator/ozone reducer based at least in part on readings of the one or more front end sensors and the one or more back end sensors.

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without parting from the spirit or scope of the present disclosure. It should be noted that like elements disclosed below are indicated by like reference numbers in the drawings.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used herein, the term “module” may refer to, be part of, or include an Application Specific Integrated Circuit (ASIC), an electronic circuit, a processor (shared, dedicated, or group) and/or memory (shared, dedicated, or group) that execute one or more software or firmware programs, a combinational logic circuit, and/or other suitable components that provide the described functionality.

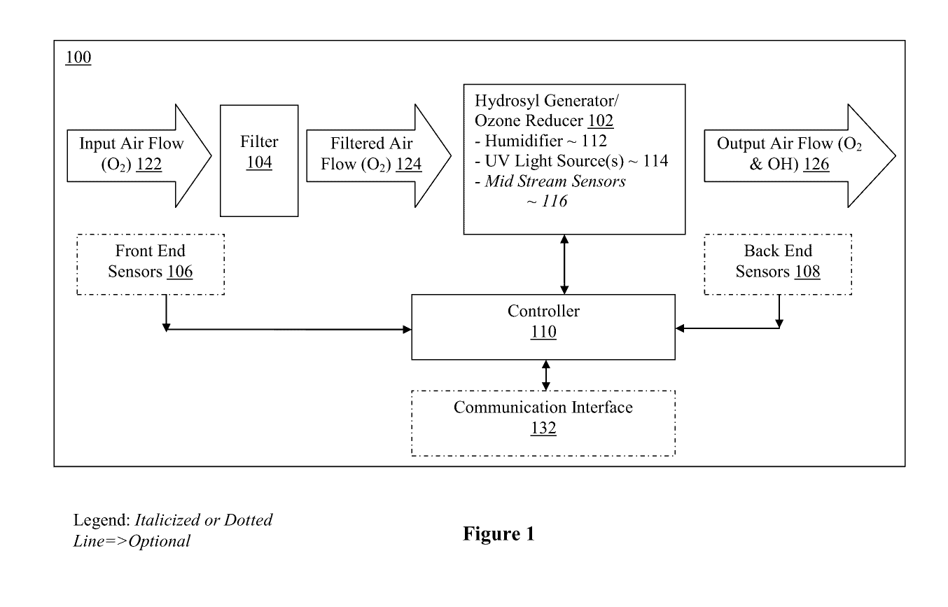

Referring now to FIG. 1, wherein a block diagram of a hydroxyl generation and/or ozone reduction system, according to the disclosed embodiments, is shown. As illustrated, in embodiments, hydroxyl generation and/or ozone reduction system 100 may include a hydroxyl generator/ozone reducer 102, filter 104, and controller 110, operatively coupled to each other. In embodiments, system 100 may further include one or more optional front end sensors 106, one or more optional back end sensors 108, and optional communication interface 132. Further, hydroxyl generator/ozone reducer 102 may include humidifier 112, one or more ultraviolet light sources 114 providing ultraviolet lights of at least two wavelengths, λ₁ and λ₂, and optional mid-stream sensors 116. As will be described in more detail below, elements 102-110 may be configured to cooperate with each other to generate an output air stream 126 with hydroxyl and/or reduced amount of ozone (including zero amount of ozone). That is, system 100 may be scaled to generate hydroxyl to clean indoor/outdoor air, reduce ozone in indoor/outdoor air, or do both. In embodiments, system 100 may generate hydroxyl with almost 0 ppb to 300 ppb of ozone.

In embodiments, front end sensors 106 may be disposed at an input end of system 100, where input air stream 122 may be provided to system 100. Front end sensors 106 may be configured to measure various attributes of input air stream 122. In embodiments, front end sensors 106 may include one or more ozone sensors, one or more ultraviolet light sensors, one or more particle sensors, one or more humidity sensors and/or one or more temperature sensors. Each ozone sensor may be configured to measure an amount of ozone in input air stream 122. Each ultraviolet light sensor may be configured to measure an amount or intensity of ultraviolet light at the input end of system 100. Each particle sensor may be configured to measure an amount of particles in input air stream 122. Each humidity sensor may be configured to measure humidity of input air stream 122. Each temperature sensor may be configured to measure temperature of input air stream 122.

Similarly, in embodiments, back end sensors 108 may include one or more ozone sensors, one or more ultraviolet light sensors, one or more particle sensors, one or more humidity sensors and/or one or more temperature sensors. Each ozone sensor may be configured to measure an amount of ozone in output air stream 126. Each ultraviolet light sensor may be configured to measure an amount or intensity of ultraviolet light at the output end of system 100. Each particle sensor may be configured to measure an amount of particles in output air stream 126. Each humidity sensor may be configured to measure humidity of output air stream 126. Each temperature sensor may be configured to measure temperature of output air stream 126.

In embodiments, communication interface 132 may be configured to enable system 100 to receive external weather and/or environmental data, and/or report its operational data. Example weather and/or environment data may include, but are not limited to, current or forecast outdoor temperature, humidity, wind speed, sunny or cloudy, precipitation level, and so forth. Example operational data may include, but are not limited to, measurements recorded by various sensors 106, 108 and/or 116, configurable operation parameters set by controller 110, such as operational parameters of humidifier 112 and at least one ultraviolet light source 114, and so forth. Examples of communication interface 132 may include, but are not limited, wired or wireless communication interfaces such as, Ethernet, Bluetooth®, WiFi, LTE, and so forth.

In embodiments, controller 110 may be configured to control hydroxyl generator/ozone reducer 102 based at least in part on readings of front end sensors 106, back end sensors 108, and/or mid stream sensors 116. For embodiments where front end sensors 106, back end sensors 108 and mid stream sensors 116 include ozone, ultraviolet light, particle, humidity and/or temperature sensors, controller 110 may be configured to control hydroxyl generator/ozone reducer 102 based at least in part on readings of these ozone, ultraviolet light, particle, humidity and/or temperature sensors. In embodiments, controller 110 may also be configured control hydroxyl generator/ozone reducer 102 based further on the external weather and/or environmental data received through communication interface 132.

In embodiments, filter 104 may include a series of filters configured to receive input air stream 122, filter it to remove e.g. particles in input air stream 122, and output filtered air stream 124. In embodiments, filter 104 may be configured to filter and remove, e.g., all particles greater than 1 micron in input air stream 122. In embodiments, filter 104 may include an electrostatic filter to filter the finer particles, and carbon ribbons to filter particles that cannot be electrostatically charged.

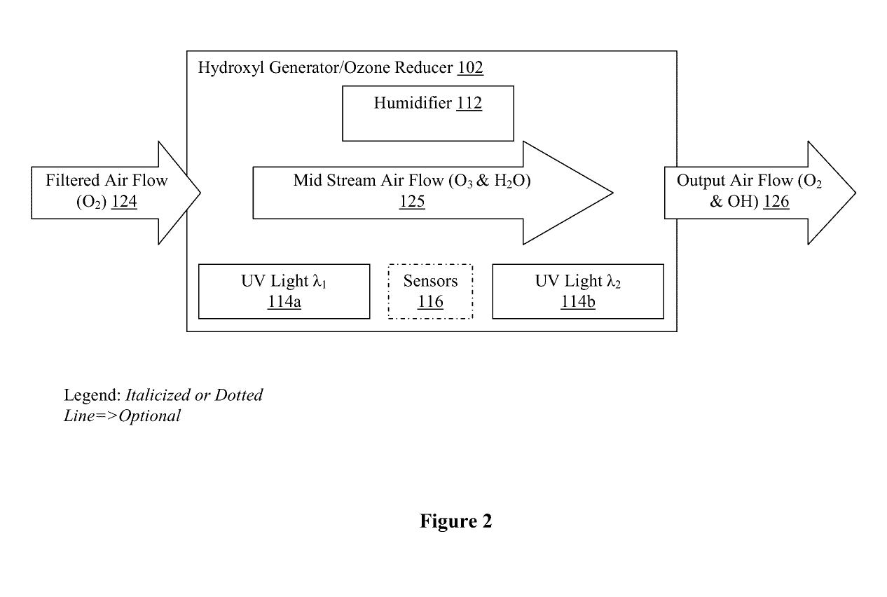

Referring now also to FIG. 2, wherein hydroxyl generator/ozone reducer 102 of FIG. 1 is illustrated in further detail, according to various embodiments. As shown and described earlier, in embodiments, hydroxyl generator/ozone reducer 102 may include humidifier 112, at least one ultraviolet light source 114 providing ultraviolet light of at least two wavelengths, λ₁ and λ₂ 114a and 114b, and optionally, one or more mid-stream sensors 116.

In particular, the one or more ultraviolet light sources may be configured to provide ultraviolet light of wavelength λ₁ of approximately 185 nm (hereinafter, simply 185 nm), and ultraviolet light of wavelength λ₂ of approximately 260 nm (hereinafter, simply 260 nm).

Whereas, one or more mid-stream sensors 116 may include various sensors configured to measure various attributes of mid stream 125. In embodiments, mid-stream sensors 116 may include one or more ozone sensors, one or more ultraviolet light sensors, one or more particle sensors, one or more humidity sensors and/or one or more temperature sensors. Each ozone sensor may be configured to measure an amount of ozone in mid-stream 125. Each ultraviolet light sensor may be configured to sense an amount or intensity of ultraviolet lights inside hydroxyl generator/ozone reducer 102 Each particle sensor may be configured to measure an amount of particles in mid stream 125. Each humidity sensor may be configured to measure humidity of mid stream 125. Each temperature sensor may be configured to measure temperature of mid stream 125. These sensor data may further complement the sensor data provided by front and back end sensors 106 and 108 to enable controller 110 to adjust attenuation of at least one light source 114 over time, in view of e.g., dirt accumulated on the surface of at least one light source 114 , or aging of at least one light source 114, and the vapor water droplets provided by humidifier 112, in view of e.g., the amount of ozone in mid stream air flow 125.

In embodiments, at least one ultraviolet light source 114 may be configured to shine 185 nm wavelength ultraviolet light on the filtered air stream 124 to generate mid stream 125 with oxygen (O₂) converted to ozone (O₃).

In embodiments, humidifier 112 may be configured to provide water vapor to interact with mid stream 125 to create humidified mid-stream 125 having a mixture of ozone (O₃) and water molecules (H₂O). In embodiments, humidifier 112 may be a steam or sonic humidifier configured to provide water vapor in very small droplets, e.g., droplets of no more than 1 cubic micron each in volume. In embodiments, humidifier 112 may be a steam humidifier configured to provide water vapor droplets of no more than 0.5 cubic micron each in volume.

In embodiments, at least one ultraviolet light source 114 may be configured to shine 260 nm wavelength ultraviolet light on the humidified mid stream 125 to generate output air stream 126 with hydroxyl (OH) and/or reduced amount of ozone (O₃) (including up to zero amount of ozone (O₃)). (O₃+H₂O→O₂+2 HO)

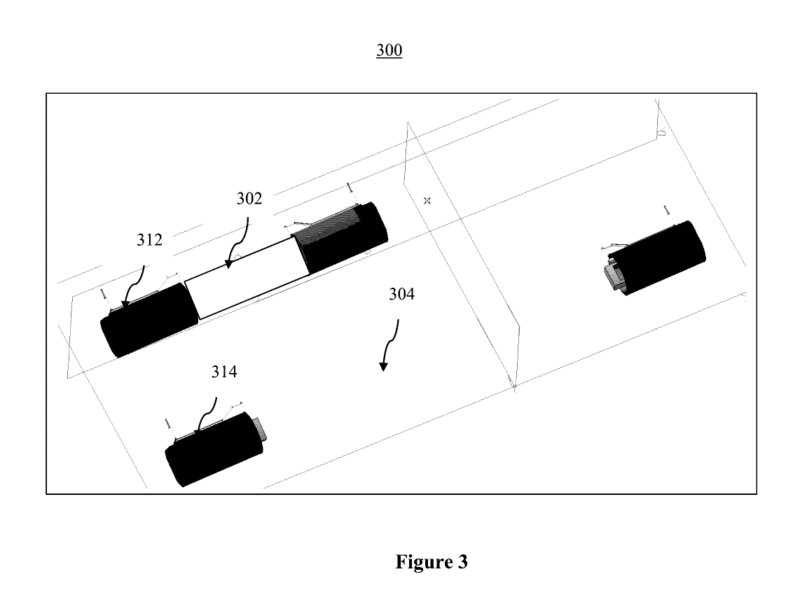

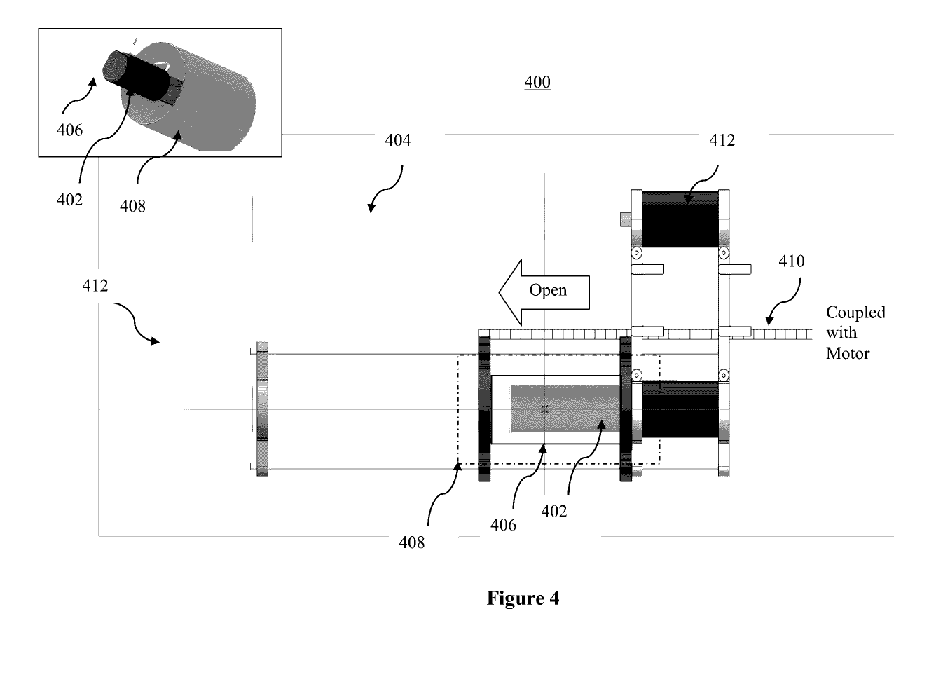

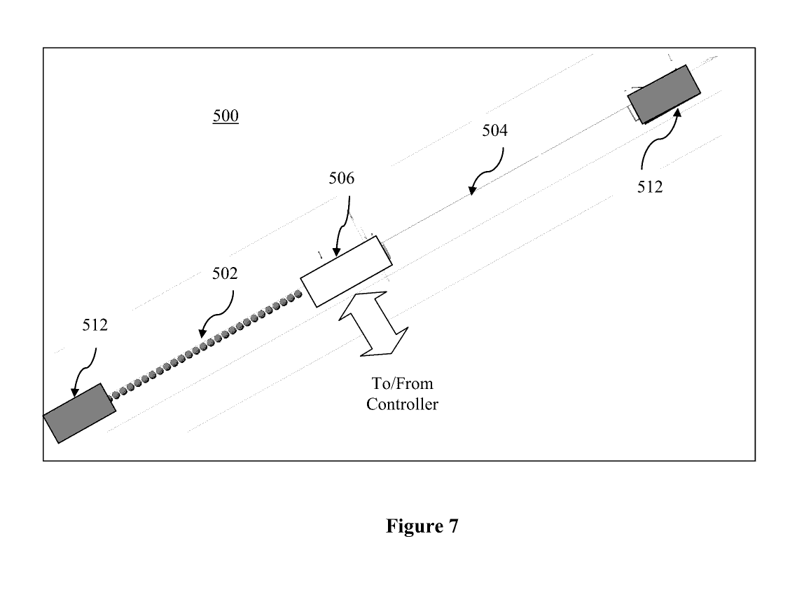

In embodiments, at least one ultraviolet light source 114 may be two light sources (as illustrated in FIGS. 3 and 7), or a combined single adjustable ultraviolet light source configured to selectively provide ultraviolet light of at least two wavelengths, in particular, ultraviolet light of 185 nm wavelength and ultraviolet light of 260 nm wavelength (as illustrated in FIGS. 4-6). These example ultraviolet light sources 114 will be further described below with references to FIGS. 3-7.

Continuing to refer to FIGS. 1 and 2, in embodiments, controller 110 may be configured to control humidifier 112 and at least one ultraviolet light source 114, based at least in part on readings of front end sensors 106, back end sensors 108, mid-stream sensors 116, and/or external weather/environmental data received through communication interface 132. For embodiments where front end sensors 106 and back end sensors 108 include ozone, ultraviolet light, particle, humidity and/or temperature sensors, controller 110 may be configured to control humidifier 112 and ultraviolet light sources 114, based at least in part on readings of these ozone, ultraviolet light, particle, humidity, temperature, air flow, and/or mid-stream sensors.

The manner in which controller 110 may control hydroxyl generator/ozone reducer 102 to produce output air stream 126 with various amount of hydroxyl and/or reduced amount of ozone (including zero amount of ozone), based at least in part on readings of the front end, back end, and/or mid-stream sensors, may be empirically determined.

In one illustrative situation, as a non-limiting example, when sensors 106/108/116 report relatively slow moving air (e.g., 1 in the scale of 1 to 10), relatively intense ultraviolet light (e.g., 10 in the scale of 1 to 10), relatively low humidity (e.g., 1 in the scale of 1 to 10), and relatively little ozone in the ambient air (e.g., 1 in the scale of 1 to 10), controller 110 may control humidifier 112 to provide a relatively small volume of vapor water droplets (e.g., 1 in the scale of 1 to 10) to generate a relatively small amount hydroxyl with very low amount (e.g., ˜10 ppb) of ozone.

In a second illustrative situation, also as a non-limiting example, when sensors 106/108/116 report relatively fast moving air (e.g., 10 in the scale of 1 to 10), relatively intense ultraviolet light (e.g., 10 in the scale of 1 to 10), relatively high humidity (e.g., 10 in the scale of 1 to 10), and moderate amount of ozone in the ambient (e.g., 5 in the scale of 1 to 10), controller 110 may also control humidifier 112 to just provide a relatively small volume of vapor water droplets (e.g., 1 in the scale of 1 to 10) to generate a moderate amount hydroxyl with very low amount (e.g., ˜10 ppb) of ozone.

In a third illustrative situation, also as a non-limiting example, when sensors 106/108/116 report moderate volume of moving air (e.g., 5 in the scale of 1 to 10), moderate ultraviolet light intensity (e.g., 5 in the scale of 1 to 10, due to degraded light source), moderate level of humidity (e.g., 5 in the scale of 1 to 10), and moderate amount of ozone in the ambient (e.g., 5 in the scale of 1 to 10), controller 110 may control humidifier 112 to provide a moderate volume of vapor water droplets (e.g., 5 in the scale of 1 to 10) to generate a moderate amount of hydroxyl with very low amount (e.g., ˜10 ppb) of ozone.

In a fourth illustrative situation, also as a non-limiting example, when sensors 106/108/116 report moderate volume of moving air (e.g., 5 in the scale of 1 to 10), moderate ultraviolet light intensity (e.g., 5 in the scale of 1 to 10, due to degraded light source), moderate level of humidity (e.g., 5 in the scale of 1 to 10), and relatively small amount of ozone in the ambient air (e.g., 1 in the scale of 1 to 10), controller 110 may control humidifier 112 to provide a moderate volume of vapor water droplets (e.g., 5 in the scale of 1 to 10) to generate a large amount of hydroxyl with very low amount (e.g., ˜10 ppb) of ozone.

Still referring to FIG. 1, in embodiments, front end sensors 106, back end sensors 108, and hydroxyl generator/ozone reducer 102, may be respectively coupled with controller 110 directly or via a shared system bus (not shown). An example of direct coupling may include the serial peripheral interface (SPI). Examples of system bus may include, but are not limited to, the I2C bus, a universal serial bus (USB), and so forth.

Referring now to FIGS. 3-7 wherein at least one ultraviolet light source of FIGS. 1 and 2, according to various embodiments, is illustrated. As shown in FIG. 3, at least one ultraviolet light source 300 may include two bulbs 302 and 304, respectively having quartz crystal to provide ultraviolet light of wavelengths 185 nm and 260 nm. The two bulbs 302 and 304 may be configured with separate electrical contacts 312 and 314 to enable the two bulbs 302 and 304 be powered on or off independent of each other, to independently provide the ultraviolet light of wavelength 185 nm ad 260 nm at the same or different points in time during operation.

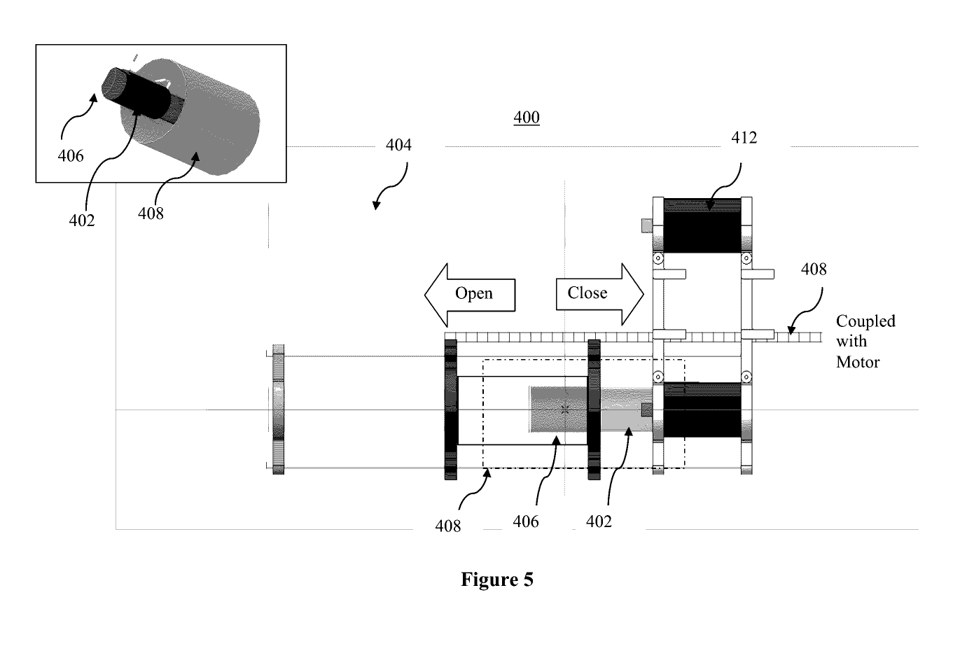

FIGS. 4-5 illustrate a single bulb arrangement 400 configured to selectively provide ultraviolet light of two wavelengths 185 nm, and 260 nm. For the embodiments, single bulb 400 has a substantially U-shaped body, where a smaller portion 402 is filled with quartz crystal to provide ultraviolet light of wavelength 185 nm, and a larger portion is filled with quartz crystal to provide ultraviolet light of wavelength 260 nm. Bulb 400 may be provided with a single set of electrical contacts 412 to power bulb 400 on and off, and a movable hood 406 that is movable between a close position and an open position to cover or expose portion 402, to control whether ultraviolet light of wavelength 185 nm is provided or not. In embodiments, mechanism 410 having a gear track coupled with a motor (not shown) may be provided and coupled with hood 406 to move hood 406 between the close and open positions to cover or expose portion 402. The gear track may be coupled to and driven by an electric, pneumatic or hydraulic motor (not shown). In embodiments, the gear track may have 35 positions at 2 mm increments over a length of about 70 mm. In alternate embodiments, in lieu of a gear track, a cable or other equivalent components may be used. FIG. 4 illustrate the movable hood 406 in a close position, and FIG. 5 illustrates the movable hood in a partially open position. Hood 406 may be formed with any material with the property of blocking the transmission of ultraviolet light of wavelength 185 nm.

Additionally, in embodiments, bulb 400 may be provided with reflector 408 encasing hood 406 and portion 402 to amplify ultraviolet light of wavelength 185 nm, when provided. The top left inserts of FIGS. 4 and 5 illustrate a zoom-in view of portion 402 with hood 406 and reflector 408. Reflector 408 may be formed with any metallic material with the property of amplifying ultraviolet light of wavelength 185 nm, e.g., aluminum.

Further, the end or bottom portion 412 of bulb 400 may be curvilinear as illustrated, or linear in other embodiments. Still further, in other embodiments, bulb 400 may have a third or more portions filled with quartz crystals configured to provide ultraviolet light of one or more other wavelengths, accompanied with one or more additional hoods to cover or expose these portions.

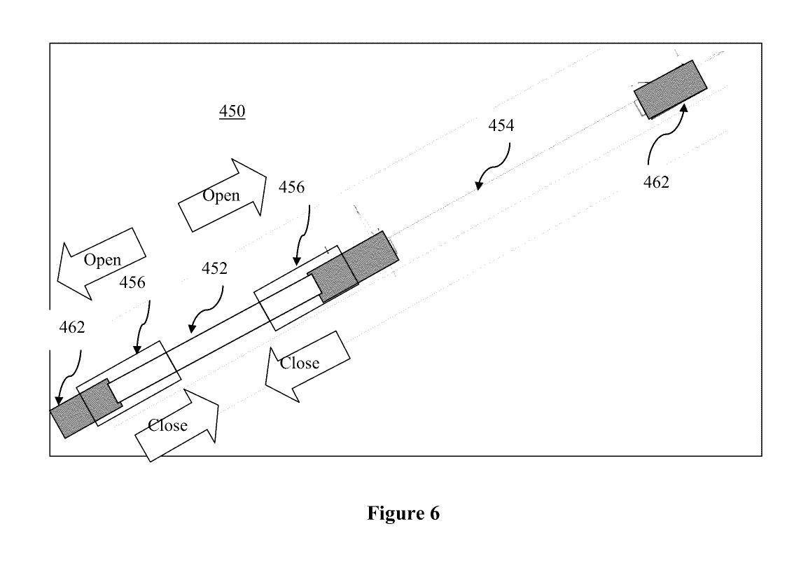

FIG. 6 illustrates yet another single bulb arrangement 450 with two portions 452 and 454 organized in a substantially linear body. Portion 452, similar to bulb 302, may be configured with quartz crystal to provide ultraviolet light of wavelength 185 nm. Portion 504 may be similar to bulb 304 configured with quartz crystal to provide ultraviolet light of wavelength 260 nm. Unlike bulb 300, but similar to bulb 400, bulb 450 may be provided with a common set of electrical contacts 462 to enable the two portions 452 and 454 be powered. Also similar to bulb 400, bulb 450 may be provided with a two part hood 456 where the two parts are movable in complementary opposite directions to cover, partially expose or fully expose portion 452, to provide or withdraw from provision of ultraviolet light of wavelength 185 nm.

FIG. 7 illustrates yet another single bulb arrangement 500 with two portions 502 and 504 organized in a substantially linear body. Portion 502, unlike bulb 302, may include a number of light emitting diodes (LED) to provide ultraviolet light of wavelength 185 nm. Portion 504 may be similar to bulb 304 configured with quartz crystal to provide ultraviolet light of wavelength 260 nm. Unlike bulb 300, but similar to bulb 400, bulb 500 may be provided with a common set of electrical contacts 512 to enable the two portions 502 and 504 be powered. Also similar to bulb 400, bulb 500 may be provided with an interface 506 to enable controller 110 to turn LED 502 on and off to provide or withdraw from provision of ultraviolet light of wavelength 185 nm, i.e., a digital hood analogous to the mechanical hood 406 or 456 of bulb 400 or 450.

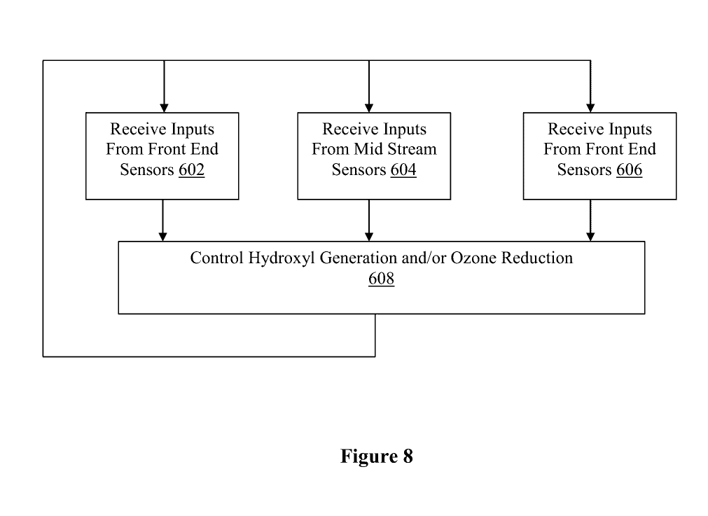

Referring now to FIG. 8, wherein an example process for generating hydroxyl and/or reducing ozone, according to various embodiments, is shown. As illustrated, process 400 for generating hydroxyl and/or reducing ozone may include operations performed at block 402-408. The operations may be performed e.g., by earlier described controller 110, which may be implemented in application specific integrated circuit (ASIC), programmable circuits (such as field programmable gate arrays (FPGA)) programmed with the operational logic, and/or software.

Process 400 may start at blocks 402, 404 and/or 406, serially or in parallel. At block 402, readings of front end sensors disposed at an input end of a hydroxyl generator/ozone reducer may be received. As earlier described, these readings may include readings of an ozone sensor, a ultraviolet light sensor, a particle sensor, a humidity sensor and/or a temperature sensor disposed at the input end of the hydroxyl generator/ozone reducer.

At block 404, readings of mid-stream sensors integrated with hydroxyl generator/ozone reducer may be received. As earlier described, these readings may include readings of one or more ultraviolet light sensors.

At block 406, readings of back end sensors disposed at an output end of a hydroxyl generator/ozone reducer may be received. As earlier described, these readings may include readings of an ozone sensor, a ultraviolet light sensor, a particle sensor, a humidity sensor and/or a temperature sensor disposed at the output end of the hydroxyl generator/ozone reducer.

From blocks 402, 404 and 406, process 400 may proceed to block 408. At block 408, hydroxyl generation and/or ozone reduction may be controlled, based at least in part on the readings of the front end, mid-stream and back end sensors. For example, as described earlier, humidification of a filtered air stream and exposure of the humidified air stream to ultraviolet lights may be controlled, based at least in part on the readings of the front end, mid-stream and back end sensors.

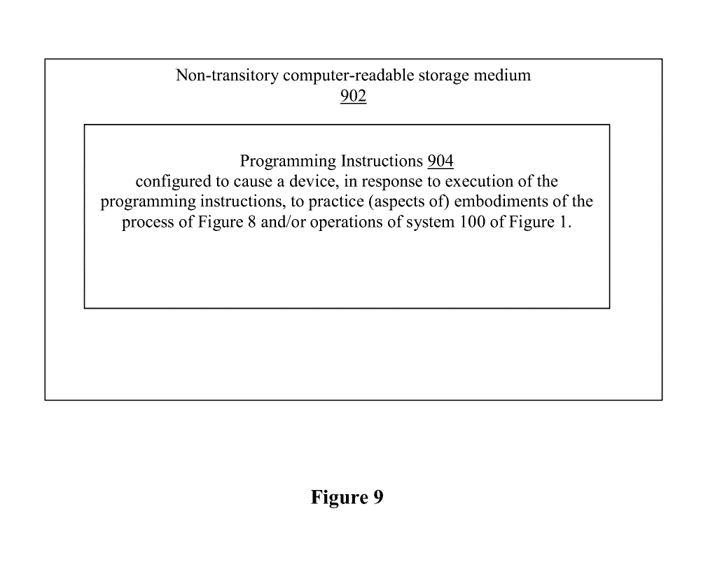

FIG. 9 illustrates an example computer-readable non-transitory storage medium that may be suitable for use to store instructions that cause an apparatus, in response to execution of the instructions by the apparatus, to practice selected aspects of the present disclosure. As shown, non-transitory computer-readable storage medium 902 may include a number of programming instructions 904. Programming instructions 904 may be configured to enable a device, e.g., controller 110, in response to execution of the programming instructions, to perform, e.g., various operations associated with controlling operations of system 100 described with references to FIGS. 1-8. In alternate embodiments, programming instructions 904 may be disposed on multiple computer-readable non-transitory storage media 902 instead. In alternate embodiments, programming instructions 904 may be disposed on computer-readable transitory storage media 902, such as, signals.

Accordingly, a novel hydroxyl generation and/or ozone reduction system has been described. It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

1. A system for generating hydroxyl or reducing ozone, comprising:
one or more front end sensors disposed at an input end of the system to measure attributes of an input air stream;
a hydroxyl generator/ozone reducer to receive the input air stream and use the input air stream to generate an output air stream with hydroxyl or reduced amount of ozone;
one or more back end sensors disposed at an output end of the system to measure attributes of the output air stream;
a controller coupled with the one or more front end sensors, the hydroxyl generator/ozone reducer, and the one or more back end sensors to control the hydroxyl generator/ozone reducer based at least in part on readings of the one or more front end sensors and the one or more back end sensors

2. The apparatus of claim 1, wherein the one or more front end sensors include an ozone sensor to sense an amount of ozone in the input air stream; wherein the one or more back end sensors include an ozone sensor to sense an amount of ozone in the output air stream; and wherein the controller is to control the hydroxyl generator/ozone reducer to generate the output air stream with less amount of ozone than the amount of ozone in the input air stream.

3. The apparatus of claim 2, wherein the hydroxyl generator/ozone reducer includes one or more ultraviolet light sources, wherein the one or more front end sensors further include an ultraviolet light sensor to sense an amount or intensity of ultraviolet light at the input end; and wherein the controller is to control the one or more ultraviolet light sources of the hydroxyl generator/ozone reducer to generate the output air stream, based at least in part on the amount or intensity of ultra violet light measured at the input end.

4. The apparatus of claim 2, wherein the hydroxyl generator/ozone reducer includes one or more ultraviolet light sources, wherein the one or more back end sensors further include an ultraviolet light sensor to sense an amount or intensity of ultraviolet light at the output end; and wherein the controller is to control one or more ultraviolet light sources of the hydroxyl generator/ozone reducer to generate the output air stream, based at least in part on the amount or intensity of ultra violet light measured at the output end.

5. The apparatus of claim 2, wherein the one or more front end sensors further include a particle sensor to sense an amount of particles in the input air stream, a humidity sensor to sense humidity of the input air stream or a temperature sensor to sense temperature of the input air stream; and wherein the controller is to control the hydroxyl generator/ozone reducer to generate the output air stream with hydroxyl based at least in part on the amount of particles, the humidity or the temperature sensed.

6. The apparatus of claim 1, wherein the hydroxyl generator/ozone reducer includes a humidifier; and wherein the controller is to control the humidifier of the hydroxyl generator/ozone reducer, based at least in part on readings of the one or more front end and hack end sensors.

7. The apparatus of claim 1, wherein the hydroxyl generator/ozone reducer includes one or more ultraviolet light sources; and wherein the controller is to control the one or more ultraviolet light sources of the hydroxyl generator/ozone reducer, based at least in part on readings of the one or more front end and back end sensors.

8. The apparatus of claim 7, wherein the hydroxyl generator/ozone reducer includes a 185 nm ultraviolet light source; and wherein the controller is to control the 185 nm ultraviolet light source of the hydroxyl generator/ozone reducer, based at least in part on readings of the one or more front end and back end sensors.

9. The apparatus of claim 7, wherein the hydroxyl generator/ozone reducer includes a 260 nm ultraviolet light source; and wherein the controller is to control the 260 nm ultraviolet light source of the hydroxyl generator/ozone reducer, based at least in part on readings of the one or more front end and back end sensors.

10. The apparatus of claim 1, wherein the hydroxyl generator/ozone reducer further includes one or more ultraviolet light sensors; and wherein the controller is to control the hydroxyl generator/ozone reducer, further based at least in part on readings of the one or more ultraviolet light sensors of the hydroxyl generator/ozone reducer.

11. The apparatus of claim 1,
wherein the one or more front end sensors include an ozone sensor, an ultraviolet light sensor, a particle sensor, a humidity sensor, and a temperature sensor;
wherein the one or more back end sensors include an ozone sensor, an ultraviolet light sensor, a particle sensor, a humidity sensor, and a temperature sensor;
wherein the hydroxyl generator/ozone reducer includes a humidifier, a 185 nm ultraviolet light source, a 260 nm ultraviolet light source and an ultraviolet light sensors; and
wherein the controller is to control the humidifier, 185 nm ultraviolet light source, and 260 nm ultraviolet light source of the hydroxyl generator/ozone reducer, based at least in part on readings of the ozone sensors, the ultraviolet light sensors, the particle sensors, the humidity sensors, and the temperature sensors.

12. A method for generating hydroxyl or reducing ozone, comprising:
receiving, by or with a controller, measured attributes of an input air stream provided to an hydroxyl generator/ozone reducer, at an input end of the hydroxyl generator/ozone reducer, with one or more front end sensors;
receiving, by or with the controller, measured attributes of an output air stream of the hydroxyl generator/ozone reducer, at an output end of the hydroxyl generator/ozone reducer, with one or more back end sensors;
controlling, by or with the controller, the hydroxyl generator/ozone reducer, based at least in part on readings of the one or more front end sensors and the one or more back end sensors, to generate the output air stream with hydroxyl or reduced amount of ozone.

13. The method of claim 12, wherein the one or more front end sensors include an ozone sensor to sense an amount of ozone in the input air stream; wherein the one or more back end sensors include an ozone sensor to sense an amount of ozone in the output air stream; and wherein controlling comprises controlling the hydroxyl generator/ozone reducer to generate the output air stream with less amount of ozone than the amount of ozone in the input air stream.

14. The method of claim 13, wherein the hydroxyl generator/ozone reducer includes one or more ultraviolet light sources,
wherein the one or more front end sensors further include an ultraviolet light sensor to sense an amount or intensity of ultraviolet light at the input end; and
wherein controlling comprises controlling the one or more ultraviolet light sources of the hydroxyl generator/ozone reducer to generate the output air stream, based at least in part on the amount or intensity of ultra violet light measured at the input eruct wherein the one or more back end sensors further include an ultraviolet light sensor to sense an amount or intensity of ultraviolet light at the output end; and wherein controlling comprises controlling one or more Ultraviolet light sources of the hydroxyl generator/ozone reducer to generate the output air stream, based at least in part on the amount or intensity of ultra violet light measured at the output end; and
wherein the one or more front end sensors further include a particle sensor to sense an amount of particles in the input air stream, a humidity sensor to sense humidity of the input air stream or a temperature sensor to sense temperature of the input air stream; and wherein controlling comprises controlling the hydroxyl generator/ozone reducer to generate the output air stream with hydroxyl based at least in part on the amount of particles, the humidity or the temperature sensed.

15. (canceled)

16. (canceled)

17. The method of claim 12, wherein the hydroxyl generator/ozone reducer includes a humidifier; and wherein controlling comprises controlling the humidifier of the hydroxyl generator/ozone reducer, based at least in part on readings of the one or more front end and back end sensors.

18. The method of claim 12, wherein the hydroxyl generator/ozone reducer includes one or more ultraviolet light sources; and wherein controlling comprises controlling the one or more ultraviolet light sources of the hydroxyl generator/ozone reducer, based at least in part on readings of the one or more front end and back end sensors.

19. The method of claim 18, wherein the hydroxyl generator/ozone reducer includes a 185 nm ultraviolet light source and a 260 nm ultraviolet light source; and wherein controlling comprises controlling the 185 nm ultraviolet light source and the a 260 nm ultraviolet light source of the hydroxyl generator/ozone reducer, based at least in part on readings of the one or more front end and back end sensors.

20. (canceled)

21. The method of claim 12, wherein the hydroxyl generator/ozone reducer further includes one or more ultraviolet light sensors; and wherein controlling comprises controlling the hydroxyl generator/ozone reducer, further based at least in part on readings of the one or more ultraviolet light sensors of the hydroxyl generator/ozone reducer.

22. The method of claim 12,
wherein the one or more front end sensors include an ozone sensor, an ultraviolet light sensor, a particle sensor, a humidity sensor, and a temperature sensor;
wherein the one or more back end sensors include an ozone sensor, an ultraviolet light sensor, a particle sensor, a humidity sensor, and a temperature sensor;
wherein the hydroxyl generator/ozone reducer includes a humidifier, a 185 nm ultraviolet light source, a 260 nm ultraviolet light source and an ultraviolet light sensors; and
wherein controlling comprises controlling the humidifier, 185 nm ultraviolet light source, and 260 nm ultraviolet light source of the hydroxyl generator/ozone reducer, based at least in part on readings of the ozone sensors, the ultraviolet light sensors, the particle sensors, the humidity sensors, and the temperature sensors.

23. One or more computer-readable storage medium having a plurality of instructions to cause a controller, in response to execution of the instructions by the controller, to practice the method of claim 12.
